(19) United States
(12) Patent Application Publication
(10) Pub. No.: US 2009/0306093 A1
(43) Pub. Date: Dec. 10, 2009

(54) METHOD FOR TREATING PATIENT HAVING BENIGN PROSTATE HYPERPLASIA

(75) Inventor: Ing-Jun Chen, Kaohsiung City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 12/135,273

(22) Filed: Jun. 9, 2008

(51) Int. Cl. A61K 31/52; A61P 13/08; A61P 13/02; A61P 15/10

(52) U.S. Cl. 514/252.16

(57) ABSTRACT

A method for treating a patient having lower urinary tract symptoms is provided. The method comprises a step of administering to the patient a therapeutically effective amount of one selected from a group consisting of a treating compound of 7-[2-[4-(2-Chlorobenzene)piperazinyl]ethyl]-1,3-dimethyl xanthine, a salt of the treating compound, a solvate of the treating compound and a combination thereof, wherein the compound has an effect on treating the benign prostate hyperplasia via a mechanism selected from a group consisting of an activation of PKG pathway, a blockage of an adrenoceptor, an opening of a potassium channel and an inhibition of PDE activity.

### FIELD OF THE INVENTION

The present invention relates to a theophylline-based compound capable of enhancing the production of cGMP, and more particularly to a compound of 7-[2-[4-(2-Chlorobenzene)piperazinyl]ethyl]-1,3-dimethyl xanthine capable of treating a patient having benign prostate hyperplasia (BPH).

### BACKGROUND OF THE INVENTION

BPH is a hormone- and age-dependent disease, resulting from a change in size, composition and function of the prostate gland, and leads to obstruction of the bladder and urethra and LUTS. The LUTS involves in an increase of prostatic cellular mass and smooth muscle tone. α₁-adrenoceptor, the predominant receptor in the human prostate, bladder neck and urethra, crucially mediates the prostate smooth muscle contraction^{[1]}. Since the best way to treating BPH is to decrease prostate size and smooth muscle tone, 5α-reductase inhibitors and selective α-androgenic blockers are thus clinically used^{[2]}. However, the proportion of α₁-adrenoceptor subtypes may be different, e.g., the ratio of α_{1A}, α_{1B} and α_{1D} subtype mRNAs being 85:1:14 and 63:6:31 in BPH and non-BPH, respectively^{[3]}. Therefore, α_{1A}/α_{1D}-adrenoceptor antagonist, tamsulosin, is preferred clinically to be useful in reducing cardiovascular side effects.

Cyclic adenosine monophosphate (cAMP) and cyclic guanosine monophosphate (cGMP) are synthesized by activation of adenylyl- and guanylyl-cyclases respectively and degraded by cyclic nucleotide phosphodiesterases (PDE)^{[4]}. In general, nitric-oxide (NO) activates soluble guanylyl cyclase (sGC), which leads to an increase in intracellular cGMP, and subsequently activates cytosolic cGMP-dependent protein kinase (PKG). Studies on smooth muscle have shown that activation of PKG may lead to the opening of large conductance Ca²⁺ activated K⁺ (BK_{Ca}) channels^{[5]} or ATP-sensitive potassium (K_{ATP}) channels^{[6]}.

Increasing evidences show that RhoA/Rho kinase (ROCK2) is involved in a variety of important physiological functions including smooth muscle contraction, cell proliferation, cell adhesion, cell migration, and various inflammatory responses^{[7]}. Particularly, activities of ROCK2 play important roles in modulating smooth muscle contraction in various tissues, including cavernosum, prostate and bladder^{[8-10]}. ROCK2 inhibitor, Y-27632, has been shown to relax rabbit and human cavernous smooth muscle and human prostate smooth muscle^{[10,11]}.

Please refer to FIG. 1, which shows a chemical structure of a compound of 7-[2-[4-(2-Chlorobenzene)piperazinyl]ethyl]- 1,3-dimethyl xanthine, denoted as KMUP-1. KMUP-1 has been investigated to relax aortic, corporeal cavernosa and tracheal smooth muscles. KMUP-1 possesses several pharmacological cGMP-enhancing activities. Therefore, it is desired to investigate whether KMUP-1 is useful in treating a patient having a BPH by accumulation of cGMP in prostate.

From the above description, it is known whether KMUP-1 involves in treating BPH has become a major problem waited to be solved. In order to overcome the drawbacks in the prior art, another pharmaceutical activity of KMUP-1 is provided. The particular design in the present invention not only solves the problems described above, but also is easy to be implemented. Thus, the invention has the utility for the industry.

### SUMMARY OF THE INVENTION

In the present invention, it is first shown that the relationship between expression of sGC/cGMP/PKG and inhibition of ROCK2 in rat prostate to certify the mentioned possibility. We investigated possible actions of KMUP-1 on prostate contraction by comparing its actions with those of PDE5A inhibitor (sildenafil), α-adrenergic blockers (doxazosin and tamsulosin) and PKG activator on rat prostate and on proliferation of human epithelial cell PZ-HPV-7 during cell growth to testify that KMUP-1-induced relaxation of the rat prostate is mediated by the activation of sGC/cGMP/PKG pathway, combined with α_{1A}/α_{1D}-adrenergic blocking activity, BK_{Ca} and K_{ATP} channels opening, and inhibition of ROCK2 and PDE.

In accordance with one aspect of the present invention, a method for treating a patient having a BPH is provided. The method comprises a step of administering to the patient a therapeutically effective amount of one selected from a group consisting of a treating compound of 7-[2-[4-(2-Chlorobenzene)piperazinyl]ethyl]-1,3-dimethyl xanthine, a salt of the treating compound, a solvate of the treating compound and a combination thereof, wherein the compound has an effect on treating the benign prostate hyperplasia via a mechanism selected from a group consisting of an, activation of PKG pathway, a blockage of an adrenoceptor, an opening of a potassium channel and an inhibition of PDE activity.

Preferably, the PKG pathway is sGC/cGMP/PKG pathway.

Preferably, the adrenoceptor is an α_{1A}/α_{1D}-adrenergic receptor with a relatively high selectivity.

Preferably, the potassium channel is one selected from a group consisting of a BK_{Ca} channel, a K_{ATP} channel and a combination thereof.

Preferably, the inhibition of PDE is combined with an inhibition of ROCK2.

In accordance with another aspect of the present invention, a method for treating a patient having a lower urinary tract symptom is provided. The method comprises a step of administering to the patient a therapeutically effective amount of one selected from a group consisting of a treating compound of 7-[2-[4-(2-iodobenzene)piperazinyl]ethyl]-1,3-dimethyl xanthine, a salt of the treating compound, a solvate of the treating compound and a combination thereof, wherein the compound has an effect on treating the benign prostate hyperplasia via a mechanism selected from a group consisting of an activation of PKG pathway, a blockage of an adrenoceptor, an opening of a potassium channel and an inhibition of PDE activity.

Preferably, the PKG pathway is sGC/cGMP/PKG pathway.

Preferably, the adrenoceptor is an α_{1A}/α_{1D}-adrenergic receptor with a relatively high selectivity.

Preferably, the potassium channel is one selected from a group consisting of a BK_{Ca} channel, a K_{ATP} channel and a combination thereof.

Preferably, the inhibition of PDE is combined with an inhibition of ROCK2.

In accordance with a further aspect of the present invention, a method for treating a patient having an erectile dysfunction is provided. The method comprises a step of administering to the patient a therapeutically effective amount of one selected from a group consisting of a treating compound of 7-[2-[4-(2-fluorobenzene)piperazinyl]ethyl]-1,3-dimethyl xanthine, a salt of the treating compound, a solvate of the treating compound and a combination thereof, wherein the compound has an effect on treating the benign prostate hyperplasia via a mechanism selected from a group consisting of an activation of PKG pathway, a blockage of an adrenoceptor, an opening of a potassium channel and an inhibition of PDE activity.

Preferably, the PKG pathway is sGC/cGMP/PKG pathway.

Preferably, the adrenoceptor is an a_{1A}/a_{1D}-adrenergic receptor with a relatively high selectivity.

Preferably, the potassium channel is one selected from a group consisting of a BK_{Ca} channel, a K_{ATP} channel and a combination thereof.

Preferably, the inhibition of PDE is combined with an inhibition of ROCK2.

The above aspects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 shows a chemical structure of KMUP-1;

FIG. 2 shows a competition diagram of KMUP-1 for [³H]-prazosin binding sites present in rat submaxillary glands (A), spleen (B), aorta (C); Ki values were calculated from the equation Kᵢ=IC₅₀/1+(³H)ligand/K_{d}; K_{d} and (³H) ligand denote the dissociation constant and the free concentration of radiolabel, respectively; antagonism by KMUP-1 (0.01-1 μM) against phenylephrine-induced contraction was performed in isolated rat prostate strip preparations (D); each value represents the mean±SEM of 8 rats;

FIG. 3(A) shows concentration-responses of KMUP-1 (0.01-100 μM), tamsulosin (0.001 nM-100 μM), sildenafil (0.01-100 μM), sildenafil (0.1 μM) combined with KMUP-1 (0.01-100 μM) or tamsulosin (0.001 nM-100 μM) on phenylephrine (10 μM) pre-contracted rat isolated prostate; each value represents the mean±SEM of 6 experiments;

FIG. 3(B) shows the effects of KMUP-1 (10 μM), tamsulosin (10 μM), sildenafil (10 μM), zaprinast (10 μM), 8-pCPT-cGMP (10 μM) and doxazosin (10 μM) on phenylephrine (10 μM) pre-contracted rat isolated prostate strips (B); each value represents the mean±SEM of 6 experiments;

FIG. 4 shows the effects of KMUP-1 (100 μM) on rat isolated prostate strips, pre-contracted with phenylephrine (10 μM) in the absence and presence of ODQ (10 μM, sGC inhibitor), RP-8pCPT-cGMPs (10 μM, PKG inhibitor), charybdotoxin (0.1 μM, large-conductance Ca²⁺-dependent K⁺ channel blockers), apamin (1 μM, small-conductance Ca²⁺-dependent K⁺ channel blockers), 4-AP (100 μM, voltage-dependent K⁺ channel blocker) and, glibenclamide (10 μM, K_{ATP} channel blocker); each value represents the mean ±SEM of 6 experiments, wherein * P<0.05 as compared with KMUP-1 100 μM (ANOVA followed by Dunnett's test).

FIG. 5 shows the effects of KMUP-1 (10, 100 μM), sildenafil (100 μM), zaprinast (100 μM) and KMUP-1 (100 μM) in the presence of ODQ (10 μM, sGC inhibitor) and SQ22536 (10 μM, adenylyl cyclase inhibitor), respectively, on accumulation of cGMP (A) and cAMP (B) in rat isolated prostate; each value represents the mean±SEM. of 8 experiments, wherein * P<0.05 as compared with the control (ANOVA followed by Dunnett's test); #P<0.05 as compared with KMUP-1 100 μM (Student's t test).

FIG. 6 shows the western analysis of the expression of sGCa1 by KMUP-1 (10, 100 μM), vehicle of KMUP-1, sildenafil (100 μM), zaprinast (100 μM) and KMUP-1 (100 μM) in the presence of ODQ (10 μM, sGC inhibitor) (A) and for the inhibition of PDE5A by KMUP-1 (1-100 μM) (B) in rat prostate; each value represents the mean±SEM of 6 experiments, wherein * P<0.05 as compared with the control (ANOVA followed by Dunnett's test) and #P<0.05 as compared with the KMUP-1 100 μM (Student's t test).

FIG. 7 shows the western analysis of the expression of PKG (A) and PKA (B) by KMUP-1 (10-100 μM), vehicle of KMUP-1, sildenafil (100 μM), zaprinast (100 μM) and KMUP-1 (100 μM) in the presence of Rp-8pCPT-cGMPs (10 μM, the specific PKG inhibitor) and SQ 22536 (10 μM, the specific adenylyl cyclase inhibitor), respectively, in rat prostate; each value represents the mean ±SEM of 6 experiments, wherein * P<0.05 as compared with the control (ANOVA followed by Dunnett's test) and ^{#}P<0.05 as compared with the KMUP-1 100 μM (Student's t test).

FIG. 8 shows the western analysis of the expression of ROCK2 by KMUP-1 (10-100 μM), vehicle-of KMUP-1 and Y-27632 (10 μM) in the presence of phenylephrine (10 μM) in rat prostate; each value represents the mean ±SEM of 6 experiments, wherein ^{#}P<0.05 as compared with the control (Student's t-test) and *P<0.05 as compared with the phenylephrine 10 μM (ANOVA followed by Dunnett's test).

FIGS. 9(A) and 9(B) show the respective effects of KMUP-1 (1-100 μM) on the PZ-HPV-7 human prostatic epithelial cells viability and cell growth by MT and Trypan blue for 24 and 48 hours;

FIGS. 9(C) to 9(G) show the cell cycle analysis of PZ-HPV-7 cells exposed to vehicle of KMUP-1, KMUP-1 (0.1-10 μM) for 48 hours; data represent the mean±SEM of 3 experiments, wherein *P<0.05 as compared with the control (ANOVA followed by Dunnett's test);

FIG. 10(A) shows the real time PCR analysis on ROCK2 gene expression in PZ-HPV-7 Cells (Control) and cells treated with KMUP-1 (1-100 μM) for 24 and 48 hours; quantitative analysis using real-time QRT-PCR of ROCK2 mRNAs in KMUP-1 (1-100 μM) treated or untreated (controls) for 24 and 48 hours.

FIG. 10(B) shows the western analysis of p21 protein expression in PZ-HPV-7 cells treated with KMUP-1 (1-10 μM) for 30 minutes, 8 hours, and 24 hours, respectively; each value represents the mean±SEM of 3 independent experiments, wherein *P<0.05 as compared with the control (ANOVA followed by Dunnett's test).

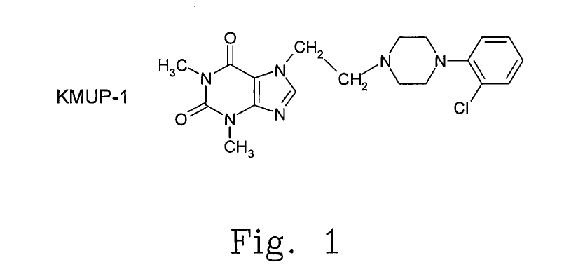

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The present invention provides a chemical compound, KMUP-1, having a pharmaceutical activity on treating BPH, easing the lower urinary tract symptoms and treating the erectile dysfunction. The detailed description for the pharmaceutical experimental results of KMUP-1 is provided as below.

Pharmacological Experiments

The synthetic method of KMUP-1 of the present invention has been disclosed in U.S. Pat. No. 6,979,687, and is not described hereafter.

Drugs: 9-(tetrahydro-2-furanyl)-9H-purin-6-amine (SQ22536), Y-27632, doxazosin, zaprinast, 8-pCPT-cGMP, RP-8-pCPT-cGMP, charybdotoxin, apamin, 4-aminopyridine (4-AP), glibenclamide, phenylephrine, and 1H-[1,2,4]oxadiazolo[4,3-a]quinoxalin-1-one (ODQ) were obtained from Sigma Chemical Co. Sildenafil citrate was supplied by Cadila Healthcare Ltd (Maninagar, India). Tamsulosin was supplied by Standard Chem. and Pharm. CO., LTD (Tainan, Taiwan). KMUP-1, artificially synthesized, was dissolved in 10% absolute alcohol, 10% propylene glycol, 2% 1N HCl and 78% distilled water. Tamsulosin and Sildenafil citrate were dissolved in distilled water.

Membrane Preparation and a_{1A}-/a_{1B}-/a_{1D}-Adrenoceptor Binding

Wistar rats were obtained from National Laboratory Animal Breeding and Research Center (Taipei, Taiwan) and housed under a constant temperature and controlled illumination. The submaxillary glands, spleen, and aorta of rats were separately homogenized for 30 seconds with a Kinematica polytron in 20 volumes of ice-cold TE buffer (10 mM Tris HCl, 1 mM EDTA, 0.1 mM ascorbic acid, pH 7.4). The homogenate was filtered under pressure through muslin and the filtrate was centrifuged at 1,000 g for 10 minutes. The supernatant was centrifuged again at 10,000 g for 12 minutes at 4° C. The second supernatant was centrifuged at 30,000 g for 15 minutes at 4° C., and the final pellet was resuspended in the assay buffer (75 mM Tris HCl, 25 mM MgCl₂, pH 7.4). Protein content was determined by Bradford's method.

[³H]-prazosin, KMUP-1 and membranes (200-300 μg) were incubated for 60 minutes at 25° C. with or without the addition of nonspecific binding agents, in a 75 mM Tris HCl buffer with 25 mM MgCl₂, making a final volume of 500 μL. In this experiment, non-specific binding was defined by use of 10 μM phentolamine. In competitive-binding experiments, the competing agent was added directly to the incubation mixture. The incubation was terminated by adding 1 mL of ice cold assay buffer followed by immediate filtration through Whatman GF/C glass fiber filters supported on a 12-port filter manifold (Whatman Int. Ltd, Springfield Mill, Maidstone, UK). The filters were immediately washed 3 times with 5 mL of ice-cold assay buffer and dried in an oven at 60° C. for 2 hours before adding 5 mL of Triton-toluene based scintillation fluid. Membrane-bound radioligand trapped in the filters was counted in a Beckman LS6500 scintillation system (Fullerton, Calif.) with an efficiency of 45%. In each experiment, nonspecifically bound radioligand agents were measured by incubating membrane protein. Specific binding was then obtained by deducting this value from the total binding of radioligand agents for each sample.

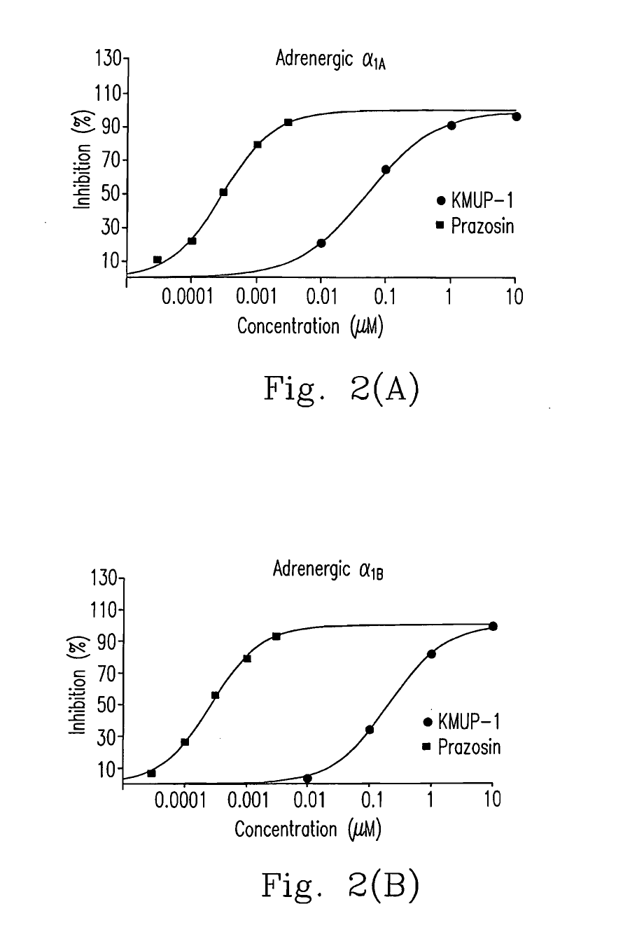

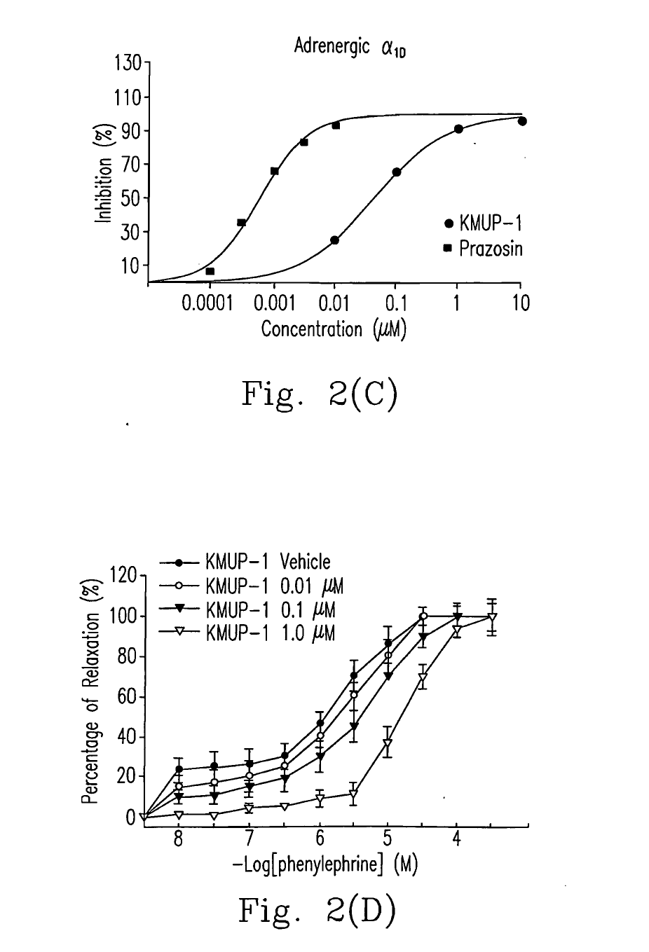

Please refer to FIGS. 2A, 2B and 2C, which shows respective competition diagrams of KMUP-1 for [³H]-prazosin binding sites present in rat submaxillary glands (A), spleen (B) and aorta (C). In this study, KMUP-1 (0.001-10 μM) inhibited [³H]-prazosin (0.1-100 nM)-produced concentration-dependent radioligand binding activity on a_{1A}-adrenoceptor, a_{1B}-adrenoceptor and a_{1D}-adrenoceptor, respectively, leading the binding affinity-curve shifted to the right. In rat submaxillary glands, spleen, and aorta, the pKᵢ values of KMUP-1 were statistically estimated to be 7.69, 6.94 and 7.69, respectively by SigmaPlot version 8.0 and SigmaStat Version 2.03 software.

Receptor affinity and binding selectivity on a₁-adrenergic receptor by KMUP-1 and previous doxazosin and tamsulosin^{[12]} are shown in Table I.

TABLE I
Receptor affinity and selectivity of a₁-adrenergic-receptor
of KMUP-1, doxazosin and tamsulosin

| | |
|---|---|
| Receptor Affinity (pKi) | Selective Ratio |

| Compound | a_{1A} | a_{1B} | a_{1D} | a_{1A}/a_{1B} | a_{1A}/a_{1D} |
|---|---|---|---|---|---|
| KMUP-1 | 7.69 | 6.94 | 7.69 | 5 | 1 |
| Doxazosin* | 8.56 | 8.98 | 8.78 | 0.4 | 1.6 |
| Tamsulosin* | 9.7 | 8.9 | 9.8 | 6.3 | 0.2 |

The data of Doxazosin* and Tamsulosin* is from the reference 25

a₁-Adrenoceptor Antagonism in Isolated Rat Prostate

Smooth muscle contractility of isolated Wistar rat prostate was used to evaluate the possible involvement of a₁-adrenoceptor antagonism by KMUP-1. KMUP-1 (0.01-1 μM) or vehicle was added to organ bath, containing the Tyrode solution, for 30 minutes after a concentration-response curve to phenylephrine (0.001-100 μM) was obtained.

Please refer to FIG. 2(D), which shows an antagonism diagram by KMUP-1 (0.01-1 μM) against phenylephrine-induced contraction performed in isolated rat prostate strip preparations. KMUP-1 (0.01-1 μM) concentration-dependently inhibited cumulatively added phenylephrine-induced contractile activities in rat isolated prostate. Concentration-response curves of phenylephrine were dose-dependently parallel-shifted to the right by KMUP-1. The estimated apparent pA₂ values and slope of regression lines for KMUP-1 on rat prostate were 7.82±0.33 and 0.80±0.53, respectively.

Isolated Prostate and Tension Measurement

The rat isolated prostate tissue was prepared as previously described with modifications^{[10]}. Briefly, Wistar rats (300-400 g) were killed with pentobarbital sodium (30 mg/kg) and the ventral part of their prostates were quickly excised. Three or four strips (10×5 mm) were mounted under 0.2-0.5 gm tension and allowed to equilibrate for 90 minutes in Tyrode solution composed of 118 mM of NaCl; 25 NaHCO₃, 4.7 KCl, 1.2 KH₂PO₄, 1.2 MgSO₄, 11 glucose, and 2.5 CaCl₂ (pH 7.4) maintained at 37° C. and aerated with 95%. O₂ and 5% CO₂.

Isometric tension was recorded with a force displacement transducer (UGO BASILE, Model 7004, Italy). After the equilibration period, the strips were primed twice by adding 10 μM of the agonist, phenylephrine, to reach 80-100% of maximum contraction. When stable constriction to phenylephrine was reached, KMUP-1 (10 μM), sildenaifil (10 μM), zaprinast (10 μM), doxazosin (10 μM), tamsulosin (10 μM) and 8-pCPT-cGMP (10 μM) were added to isolated prostate strips for 1 hour to examine whether PDE inhibition and a-adrenergic blockade could provide relaxation activity on rat prostate. The relaxation on rat prostate by KMUP-1 (0.01-100 μM) and tamsulosin (0.001 nM-100 μM) were also tested by their combination with sildenafil (0.1 μM), respectively. In isolated prostate strips, while stable constriction to phenylephrine was reached, sildenafil (0.1 μM) was pre-incubation for 30 minutes, before the cumulative addition of KMUP-1 (0.01-100 μM) or tamsulosin (0.001 nM-100 μM) to isolated prostate strips. In another experiment, to examine the possible mechanism of action of KMUP-1, rat prostates were pretreated with ODQ (1H-[1,2,4]oxadiazolo[4,3-a]quinoxalin-1-one, 10 μM, sGC inhibitor), RP-8pCPT-cGMPs (10 μM, PKG inhibitor), glibenclamide (10 μM, K_{ATP} channel blocker), 4-AP (4-aminopyridine, 100 μM, voltage-dependent K⁺ channel blocker), apamin (1 μM, Ca²⁺-dependent K⁺ channel blocker) and charybdotoxin (ChTX) (0.1 μM, Ca²⁺-dependent K⁺ channel blocker) for 30 minutes before addition of KMUP-1 (100 μM).

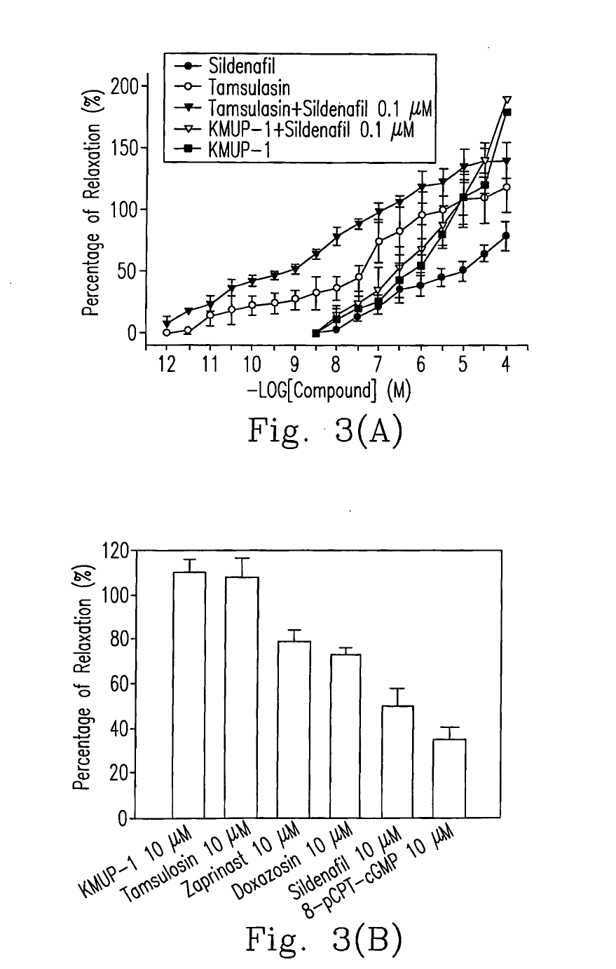

Please refer to FIG. 3(A), which shows the concentration-responses of KMUP-1 (0.01-100 μM), tamsulosin (0.001 nM-100 μM), sildenafil (0.01-100 μM), sildenafil (0.1 μM) combined with KMUP-1 (0.01-100 μM) or tamsulosin (0.001 nM-100 μM) on phenylephrine (10 μM) pre-contracted rat isolated prostate. It is found that the cumulative concentration-response curves of KMUP-1, sildenafil, tamsulosin and combination of sildenafil and KMUP-1 or tamsulosin against α-adrenceptor agonist phenylephrine-induced contraction in rat isolated prostate. KMUP-1 (100 μM), tamsulosin (100 μM) and sildenaifil (0.1 μM) were found to have 179.00±5.35, 118.00±10.79, 21.50±2.12 and 78.00±8.02% relaxation ability, respectively.

Please refer to FIG. 3(B), which shows the effects of KMUP-1 (10 μM), tamsulosin (10 μM), sildenafil (10 μM), zaprinast (10 μM), 8-pCPT-cGMP (10 μM) and doxazosin (10 μM) on phenylephrine (10 μM) pre-contracted rat isolated prostate strips. According to the data in FIG. 3(B), KMUP-1 and tamsulosin at 10 μM could relax the prostate to 110.00±5.09 and 107.50±6.76%, respectively; zaprinast, doxazosin, sildenaifl and 8-pCPT-cGMP at 10 μM, compared to nontreatment control, could relax to 79.00±5.00, 73.00±2.8, 50.00±6.37 and 35.00±4.40%, respectively.

Furthermore, please refer to FIG. 3(A) again, wherein the combination of tamsulosin (100 μM) and sildenafil (0.1 μM) displayed more relaxation activity (140.00±10.14%) than tamsulosin (100 μM) alone; in contrast, the combination of KMUP-1 (100 μM) and sildenafil (0.1 μM) displayed 190.00±10.14% relaxation activity on rat prostate. It is found that sildenafil can enhance the relaxation activity of tamsulosin more than KMUP-1 can do on rat prostate. Although the potency of KMUP-1 is less than that of tamsulosin, the maximal efficacy of KMUP-1 at 100 μM is 179.00±5.35%, which is greater than that of tamsulosin.

Determination of cGMP/cAMP

Isolated prostate strips from Wistar rats were prepared to determine prostate levels of cGMP and cAMP. Briefly, prostate strips were respectively incubated with KMUP-1 (10-100 μM), sildenafil (100 μM), and zaprinast (100 μM) in 10 mL Tyrode solution aerated with 95% O₂ and 5% CO₂ for 1 hour. To examine the possible mechanisms of action of KMUP-1, the rat prostate was pretreated with ODQ (10 μM, sGC inhibitor) and SQ22536 (9-(tetrahydro-2-furanyl)-9H-purin-6-amine, 10 μM, adenylyl cyclase inhibitor) for 30 minutes before KMUP-1 was added. After the incubation, the prostate tissues were extracted with 1 mL of 0.1 N HCl. The extracts were then lyophilized for a further measurement of cyclic GMP or cyclic AMP of each sample, using commercially available radioimmunoassay kits (Amersham Pharmacia Biotech, Buckinghamshire, England).

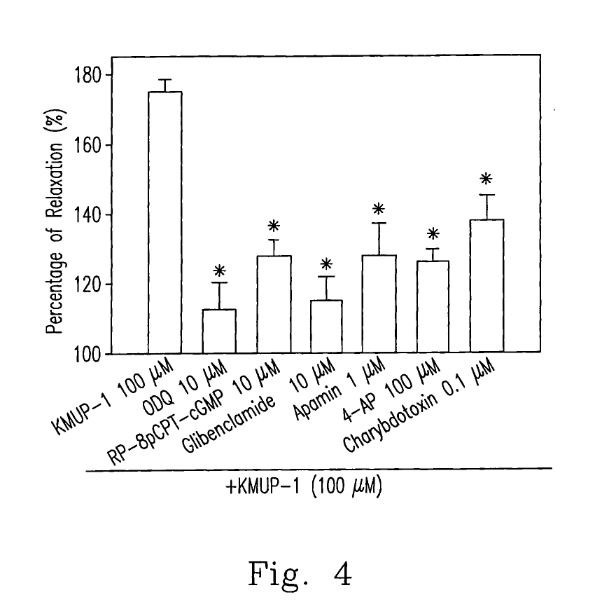

Please refer to FIG. 4, which shows the effects of KMUP-1 (100 μM) on rat isolated prostate strips, pre-contracted with phenylephrine (10 μM) in the absence and presence of ODQ (10 μM, sGC inhibitor), RP-8pCPT-cGMPs (10 μM, PKG inhibitor), charybdotoxin (0.1 μM, large-conductance Ca²⁺-dependent K⁺ channel blocker), apamin (1 μM, small-conductance Ca²⁺-dependent K⁺ channel blocker), 4-AP (100 μM, voltage-dependent K⁺ channel blocker) and glibenclamide (10 μM, K_{ATP} channel blocker). The relaxations of rat prostate strips elicited by KMUP-1 (100 μM) were significantly inhibited by a pretreatment with RP-8pCPT-cGMPs (10 μM) and ODQ (10 μM), respectively. Results showed that a pre-incubation of rat prostate with ODQ and RP-8pCPT-cGMPs reduced the relaxation, caused by KMUP-1 (100 μM), to 112.50±7.84 and 128.00±4.41%, in preparations pre-contracted with phenylephrine (10 μM), respectively.

Moreover, the relaxation of prostate, pre-contracted with phenylephrine (10 μM) by KMUP-1 (100 μM), was attenuated by K⁺ channel blockers, such as glibenclamide (115.00±7.07%), 4-AP (122.00±3.74%) and apamin (126.00±9.09%) and charybdotoxin (138.00±7.01%), respectively.

Functional studies have shown the a_{1A}-adrenoceptor subtype able to mediate prostate smooth muscle contraction in humans and animals. Both functional and radioligand binding studies have found terazosin and doxazosin to have potent cardiovascular effects and to have greater affinities for a_{1B}-adrenoceptor than a_{1A}-adrenoceptor^{[12]}. Some studies have demonstrated that highly selective a_{1A}-adrenoceptor antagonists lack cardiovascular side effects on LUTS^{[12]}. a_{1D}-adrenoceptor has also been found to be significantly up-regulated in rat obstructed bladder^{[13]}. In addition, some studies have demonstrated that a_{1D}-adrenoceptor selective antagonists ameliorate irritative symptoms^{[14]}. Although the exact role of a_{1D}-adrenoceptor in the prostate remains unclear, we cannot ignore their importance. Selectivity for the a_{1A} and/or a_{1D} subtypes over the a_{1B} subtype is referred to as “uroselectivity”^{[12]}.

Table I shows that the subtype selection of KMUP-1 is a_{1A}=a_{1D}>a_{1B}. Therefore, KMUP-1 might be able to serve as an uroselective antagonist, similar to tamsulosin, for the treatment of BPH.

In order to investigate whether KMUP-1 is involved in PKG, sGCa1 and PKA protein expressions and the relaxation activity of rat prostate, the following experiments are designed.

Protein Extraction and Western Blotting

Prostate strips were incubated with KMUP-1 (10-100 μM), sildenafil (100 μM) and zaprinast (100 μM) in 10 mL Tyrode solution aerated with 95% O₂ and 5% CO₂ for 1 hour. To examine the possible mechanisms of KMUP-1 action, the rat prostate were pretreated with ODQ (10 μM), SQ22536 (10 μM) and RP-8pCPT-cGMPs (10 μM) for 30 minutes prior to the addition of KMUP-1 (100 μM).

In order to examine the expression of Rho kinase in prostate, the prostate strips were pre-incubated with phenylephrine (10 μM) for 30 minutes. After a pre-treatment of phenylephrine, KMUP-1 (10-100 μM) and Y-27632 (10 μM) were further incubated for 30 minutes. After the incubation time, the prostate were extracted with protein extraction reagent (Pierce Biotechnology, Inc., Rockford, Ill., USA.). The PZ-HPV-7 cells were incubated with and without KMUP-1 (0.1-10 μM) at 30 minutes, 8 and 24 hours. They were then washed rapidly with ice-cold PBS, incubated with protein extraction reagent, and scraped from the plates. The prostate tissues and cells were sonicated three times for 10 second each time and centrifuged at 13,000 rpm at 4° C. for 30 minutes. The protein concentrations of supernatants were determined, using bovine serum albumin as the standard. Tissue and cell extract protein were then boiled at a ratio of 4:1 with sample buffer (100 mM Tris, pH 6.8, 20% glycerol, 4% SDS, and 0.2% bromphenol blue). 40-60 μg protein was used in this experiment. Electrophoresis was performed using 10 or 12% SDS-polyacrylamide gel electrophoresis and then transferred to nitrocellulose membranes (Millipore Corporation, Billerica, Mass.). The membrane was blocked with Tris-buffered saline (20 mM Tris and 137 mM NaCl, pH 7.6) containing 0.1% Tween 20 (TTBS) and 5% non-fat milk at room temperature for 1 hour, washed with TTBS, and then incubated overnight at 4° C. in the appropriate primary antibody of PKG (Santa Cruz, 1:200), sGCa1 (Santa Cruz, 1:200), PKA (Abcam, 1:1000), ROCK2 (Upstate, 1:1000), and PDE5A (BD Biosciences, 1:1000), p21 (Upstate, 1:1000). The membranes were washed in TTBS before being incubated with horseradish peroxidase-conjugated antibody against mouse, goat, or rabbit IgG (Santa Cruz, 1:1000) for 1 hour. The membrane was then washed in TTBS and developed with the enhanced chemiluminescence (ECL) for the detection of the specific antigen. The intensity of the bands was measured by densitometry.

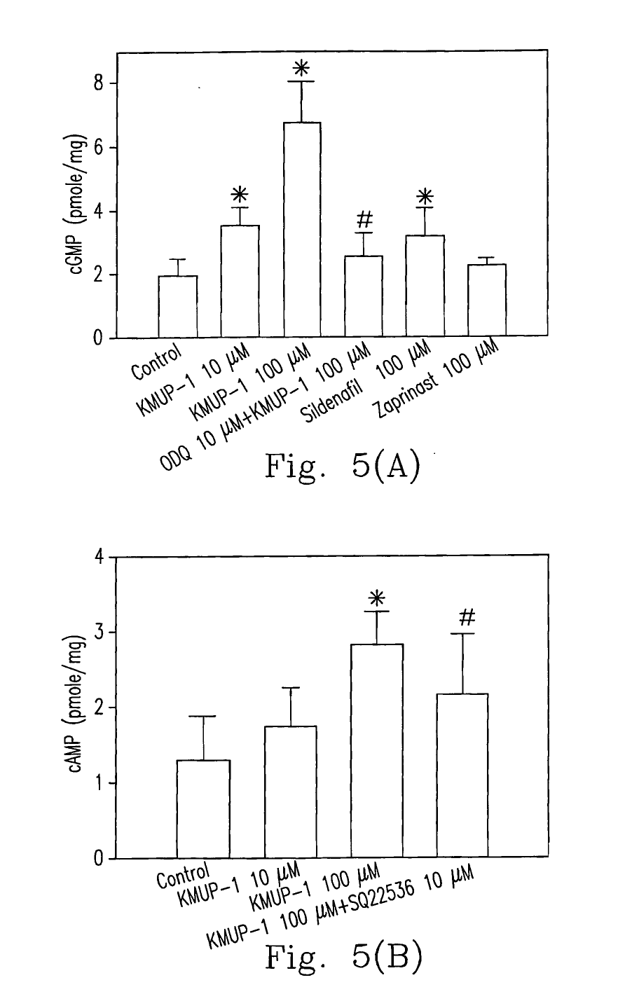

Please refer to FIGS. 5(A) and 5(B), which show the effects of KMUP-1 (10, 100 μM), sildenafil (100 μM), zaprinast (100 μM) and KMUP-1 (100 μM) in the presence of ODQ (10 μM) and SQ22536 (10 μM), respectively, on respective accumulations of cGMP and cAMP in rat isolated prostate. It is found that the basal release of cAMP and cGMP in non-treatments of rat prostate strips are 1.28±0.60 and 1.98±0.51 pmol mg⁻¹ protein, respectively. KMUP-1 (10, 100 μM) significantly increases cGMP levels (3.50±0.58, 7.50±0.79 pmol mg⁻¹ protein). The accumulation of cGMP by KMUP-1 (100 μM) alone and in the presence of ODQ (10 μM) are 7.50±0.79 and 2.54±0.73 pmol mg⁻¹ protein, respectively. It reveals that the raised levels of cGMP induced by KMUP-1 (100 μM) are inhibited by a pretreatment with ODQ (10 μM). Sildenafil (100 μM) and zaprinast (100 μM) also increase cGMP levels (3.20±0.61 and 2.23±0.24 pmol mg⁻¹ protein).

Moreover, KMUP-1 (10, 100 μM) significantly increases cAMP levels (1.74±0.51, 2.82±0.42 pmol mg⁻¹ protein) and which are inhibited by a pretreatment with SQ22536 (10 μM), respectively. The accumulation of cAMP by KMUP-1 (100 μM) alone and in the presence of SQ22536 (10 μM) are 2.82±0.42 and 2.16±0.79 pmol mg⁻¹ protein, respectively.

As shown in FIG. 3(A), the relaxation ability by KMUP-1 not only returns phenylephrine-induced contractility to near basal level at 10 μM, indicating a_{1A}/a_{1D}-adrenceptor blockade-derived relaxing activity, and further downwards reduces the level to 179.00±5.3% at 100 μM, indicating the involvement of cGMP-derived enhancing activity on relaxation. This involvement is confirmed by inhibition with ODQ and RP-8pCPT-cGMP, respectively (FIG. 4). It is thus suggested that KMUP-1 has an action in relaxing the rat prostate.

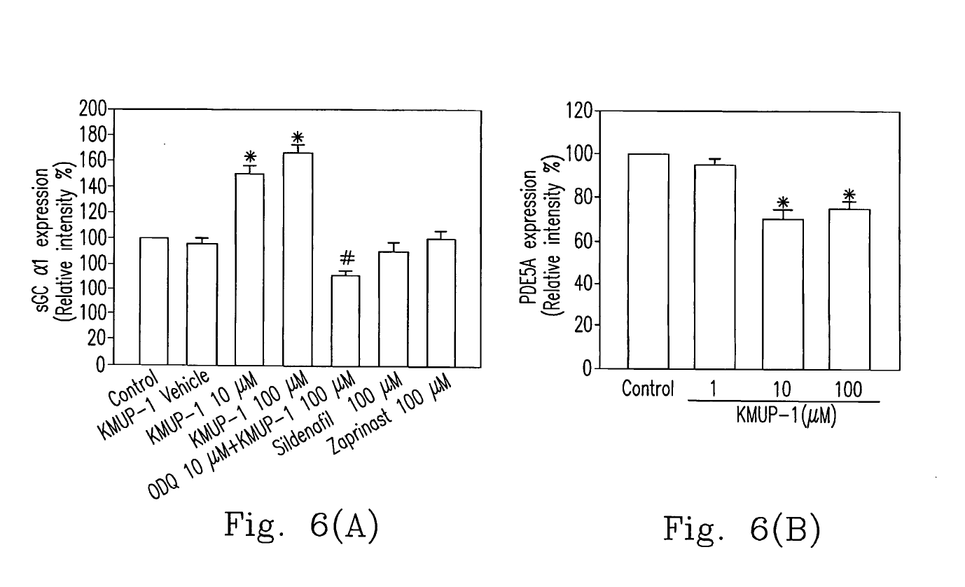

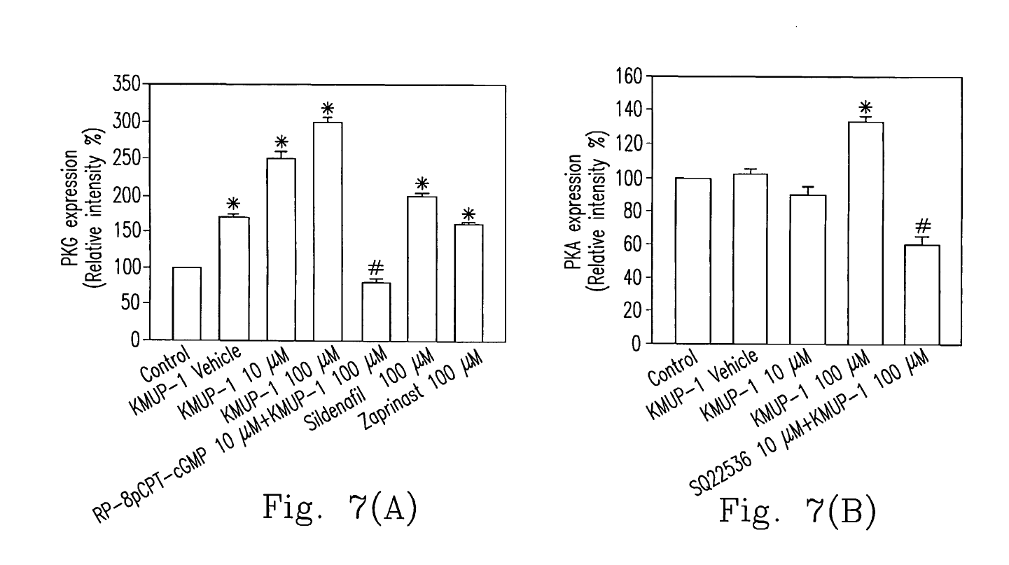

sGC, consisting of a1 and β1 subunits, is a target enzyme of endogenous NO, and it converts GTP to cGMP, resulting in smooth muscle relaxation^{[15]}. Some studies have detected the expression of PDE4, PDE5 and PDE11 in human prostate^{[16, 17]}. The current study has revealed that KMUP-1 strongly induces sGCa1 expression rather than sGCβ1. Please refer to FIGS. 6(A), 6(B) and FIGS. 7(A), 7(B), which show the results of sGCa1, PDE5A, PKG and PKA expression measured by western blot, respectively.

Please refer to FIGS. 6(A) and 6(B), which show the sGCa1 expression in the respective conditions of KMUP-1 (10, 100 μM), vehicle of KMUP-1, sildenafil (100 μM), zaprinast (100 μM) and KMUP-1 (100 μM)/ODQ (10 μM) and show the inhibition of PDE5A by KMUP-1 (1-100 μM) in rat prostate. As shown in FIG. 6, KMUP-1 (10-100 μM) most potently induces the expression of sGCa1 and also mildly inhibits the expression of PDE5A. However, zaprinast (100 μM) and sildenafil (100 μM) do not significantly induce the expression of sGCa1.

Please refer to FIGS. 7(A) and 7(B), which show the PKG and PKA protein expression under the respective conditions of KMUP-1 (10-100 μM), vehicle of KMUP-1, sildenafil (100 μM), zaprinast (100 μM), KMUP-1 (100 μM)/Rp-8pCPT-cGMPs (10 μM) and KMUP-1 (100 μM)/SQ 22536 (10 μM), respectively, in rat prostate by running the western blot. FIG. 7(A) shows that KMUP-1 (10, 100 μM), sildenafil (100 μM) and zaprinast (100 μM) all significantly induce the expression of PKG.

In addition, it is found that a pretreatment of ODQ (10 μM) and RP-8-pCPT-cGMPs (10 μM) for 30 minutes significantly reduce the expression of sGCa1 and PKG in the presence of KMUP-1 (100 μM), respectively. As shown in FIG. 7(B), KMUP-1 (100 μM) significantly induces the expression of PKA, which is reversed by SQ22536 (10 μM).

It is suggested that the activation of sGC by KMUP-1 plays an important role in relaxing rat prostate. This study further confirms that PDE5 inhibition, PKG activation and a-adrenergic blocking co-regulate or independently regulate the prostate smooth muscle tone in rats. The relaxation of prostate by KMUP-1 brings out not only sGC activation but also PDE inhibition (FIG. 6). Up-regulation of sGC and inhibition of PDE by KMUP-1 indicates the ability to increase the expression of PKG and PKA. However, inhibition of PDE5 by NO-sensitive sGC activator, was described to prevent from tolerance in hemodynamic effects, resulting from a negative feedback in NO/cGMP signaling^{[18, 19]}. This fact indicates the use of KMUP-1, taking advantage of sGC activation and PDE5A inhibition, might be beneficial in this regard.

In addition, previous studies have demonstrated that PKG inhibits the contractility through an activation of K_{ATP} channels in both human-cultured prostatic stromal cells and in prostate tissue^{[20, 21]}. In human cultured prostatic stromal cells, the elevation of intracellular cAMP stimulates PKA to open BK_{Ca} channels. In this invention, it is also shown that KMUP-1 can potently induce the expression of PKG and PKA (FIG. 7). The possible mechanism of action is suggested that cross-activation of PKA and PKG contributing to prostate relaxation by KMUP-1 through the activation of BK_{Ca} and K_{ATP} channels.

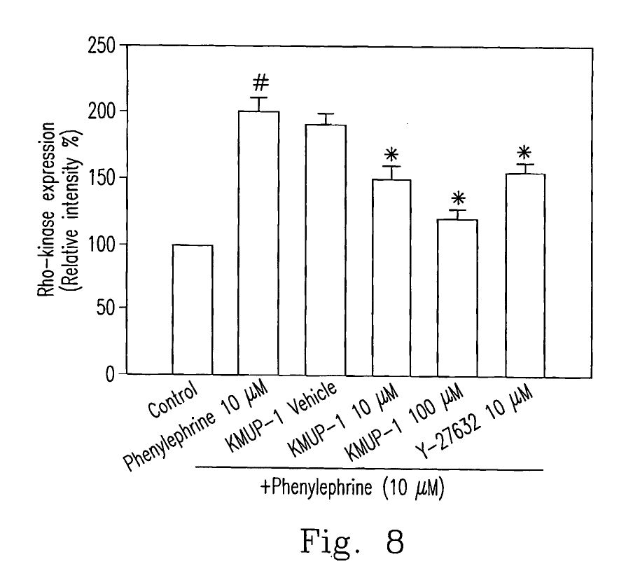

Please refer to FIG. 8, which shows the Rho kinase expression under the respective conditions of KMUP-1 (10-100 μM), vehicle of KMUP-1 and Y-27632 (10 μM) in the presence of phenylephrine (10 μM) in rat prostate. The result indicated that phenylephrine (10 μM, α-adrenoceptor agonist) strongly activated the expression of Rho kinase. However, both KMUP-1 (10, 100 μM) and Y-27632 (10 μM) protected against phenylephrine-induced Rho kinase expression.

In order to investigate whether KMUP-1 can protect against the growth of human prostate epithelial cells, the following experiments are designed.

Cell Proliferation

PZ-HPV-7, a human prostate epithelial cell line derived from normal peripheral zone tissue and transfected with human papillomavirus type 18, was obtained from the American Type Culture Collection (ATCC). Cells were grown in K-SFM medium supplemented with 100 U/mL penicillin, 100 gm/L, streptomycin 5 ng/ml, EGF human recombinant and 50 g/mL BPE in 75-cm² flasks at 37° C. in a humidified atmosphere of O₂/5% and CO₂/95% air. The culture medium was renewed every 2 days and cells were sub-cultured every 6 days. For the experiments, cells were seeded in 24-well plates at a density of 1×10⁴ cells/well. After 24 hours and 48 hours, when cells were 80% confluent, KMUP-1 (1-100 μM) was added to the 24-well plates. Cell proliferation was measured using MTT assay (3-[4,5-dimethylthiazol-2-yl]-2,5-diphenyl-tetrazolium bromide, Sigma). The trypan blue (3-[4,5-dimethylthiazol-2-yl]-2,5-diphenyl-tetrazoliumbromide) exclusion assay was performed to determine the proportion of living and dead cells in 24 hours and 48 hours after treatment. After 24 hours and 48 hours of treatment with KMUP-1 (1-100 μM), attached cells were collected by trypsinization. Cells were counted in a hemocytometer using equal volume of cell suspension and trypan blue solution (0.2% in PBS).

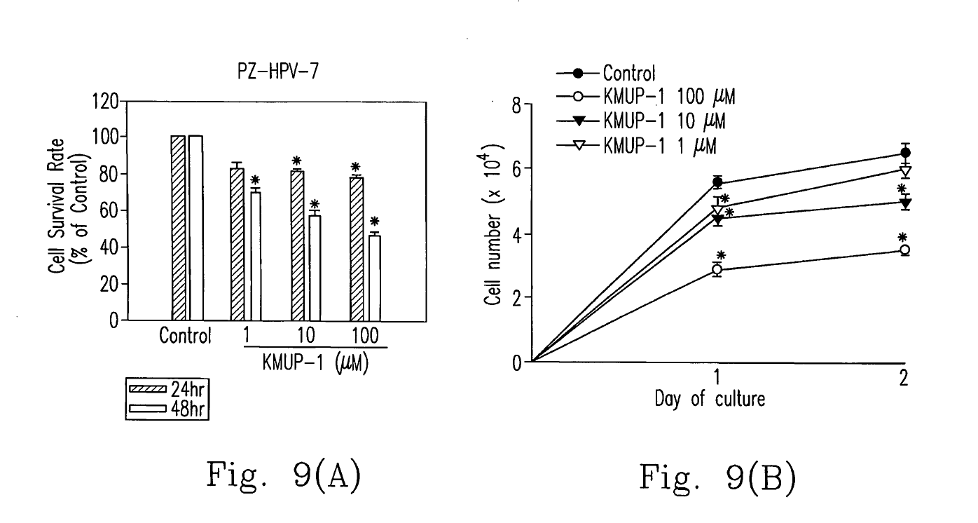

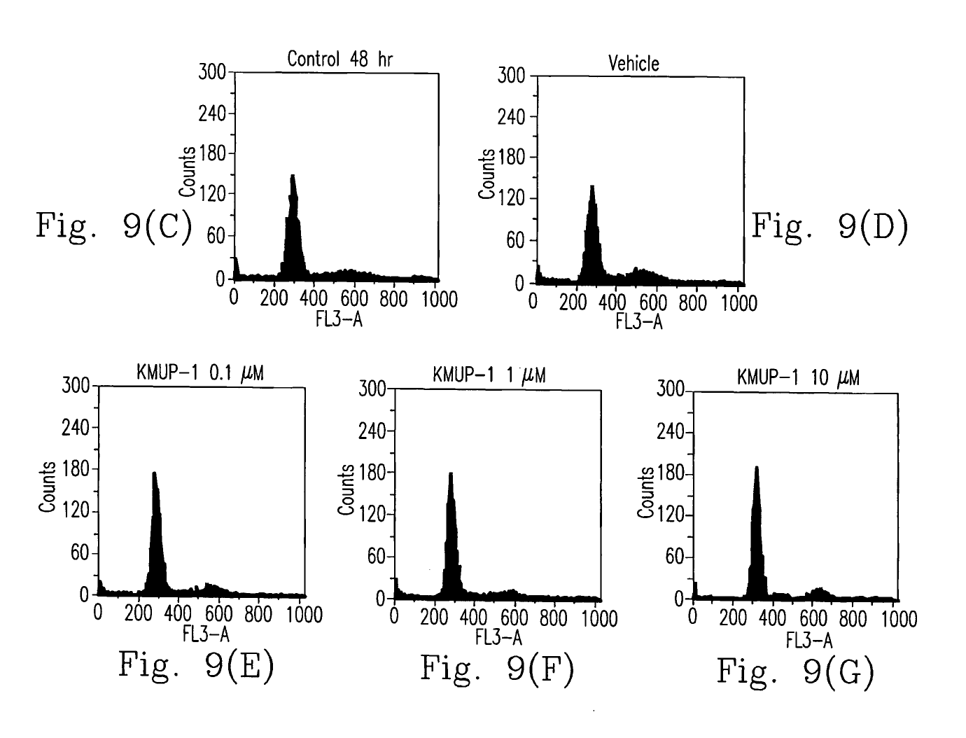

Please refer to FIGS. 9(A) and 9(B), which show the respective effects of KMUP-1 (1-100 μM) on the PZ-HPV-7 human prostatic epithelial cells viability and cell growth by MTT and Trypan blue for 24 and 48 hours. PZ-HPV-7 cells were exposed to vehicle of KMUP-1, KMUP-1 (0.1-10 μM) for 48 hours. As shown in FIG. 9(A), it is found that KMUP-1 inhibits the growth of PZ-HPV-7 in a time- and dose-dependent manner. After treatment for 24 hours, KMUP-1 (1, 10, 100 μM) inhibits the cell growth down to 82.00±3.03, 80.03±1.52 and 79.00±1.35% of control. KMUP-1 (1-100 μM) inhibits cell growth down to 70.00±2.23, 56.00±2.72, and 47.00±1.8% of control after treatment for 48 hours, respectively. Trypan blue assay also showed the dose- and time-dependent inhibition of KMUP-1 on PZ-HPV-7 cell growth.

BPH is believed to be secondary to an increased cell proliferation of both stromal and epithelial prostate cells^{[22]}. Some studies have shown that vadenafil, tadalafil, NO donors and sildenafil display anti-proliferation activity in human prostate smooth muscle cells isolated from BPH patients^{[23, 24]}. As shown in FIGS. 9(A) and 9(B), KMUP-1 can inhibit human prostatic epithelial cells growth. Therefore, KMUP-1, similar to the PDE5 inhibitors vadenafil, tadalafil, sildenafil, can display the anti-proliferation activity to hinder the proliferation of prostate cells.

Cell Cycle Distribution

PZ-HPV-7 cells were incubated for 2 days with or without KMUP-1 (0.1-10 μM), after trypsinized for 10 minutes with 0.5% trypsin supplemented with 5.3 mM EDTA in HBBS. The cell suspension was then centrifuged at 200 g for 5 minutes at 4° C., resuspended in 1 mL ice-cold HBSS and fixed by the drop-wise addition of 2 mL of ice-cold methanol followed by continuously shaking. The processed cells were kept on ice for 30 minutes and then cells were collected by centrifugation at 90 g for 5 min and stained with propidium iodide (50 μg/mL, Sigma). RNase A (Promega, Madison, Wis.) was then added to a final concentration of 100 U/mL. Samples were stored in the dark for at least 30 minutes before an analysis on a Coulter Epics XL-MCL flow cytometer. Flow cytometry analysis was performed on a Coulter Epics XL-MCL.

Please refer to FIGS. 9(C) to 9(G), which show the cell cycle analysis of PZ-HPV-7 human prostatic epithelial cells at different KMUP-1 concentrations. The cell cycle distribution is assessed after the KMUP-1 treatment for 48 hours at various concentrations. Cells are treated with KMUP-1 (0.1, 1, 10 μM) for 48 hours and result in 7±1.54, 10±2.07, 12.00±0.88% increase of G₀/G₁ phase, compared with vehicle control. As a whole, KMUP-1 arrests the cell cycle at G₀/G₁ phase of PZ-HPV-7 cells.

RNA Isolation and cDNA Synthesis

PZ-HPV-7 cells were incubated for 2 days with or without KMUP-1 (1-100 μM). After the incubation, total RNA is isolated by processing the PZ-HPV-7 cells with TRI REAGEANT (Molecular Research Center, INC., Cincinnati, Ohio, USA) and 1-bromo-3chloropropane (BCP). Isopropanol was used to precipitate the isolated RNA; ethanol (75%) was used to wash the unwanted solution. The samples were then air-dried for 15 minutes at the room temperature. The pellet was re-suspended in 20 μL of DEPC-water (Ambion). After the RNA isolation, total RNA (2 μg) was used for the reverse transcription to cDNA by High-Capacity cDNA Reverse Transcription Kits (Applied Biosystems).

Quantitative Reverse Transcription-PCR

Quantitation of Rho kinase and an internal reference gene (GAPDH) were done by fluorescence-based real-time detection (ABI PRISM 7900HT Sequence Detection System (Taqman); Applied Biosystems, Foster City, Calif.). The probe sequences used are listed in Table II. The PCR reaction mixture consisted of 1× TaqMan Master Mix, 1× Probe/primers Assay Mix, 100 ng cDNA, and water containing a reference dye, to a final volume of 20 μL (all reagents from Applied Biosystems). Cycling conditions were 50° C. for 2 minutes, 95° C. for 10 minutes, followed by 40 cycles at 95° C. for 15 second and 60° C. for 1 minute. Gene expression values (relative mRNA levels) are expressed as ratios (differences between the Ct values) between the genes of interest and an internal reference gene (GAPDH) providing a normalization factor for the amount of RNA isolated from cells. This analysis was performed by 7900 HT SDS 2.2.1 software.

TABLE II
Quantitative RT-PCR: Probes sequences utilized
ABI Gene expression assays

| Gene | | | Amplicon |
|---|---|---|---|
| symbol | GENBANK accession # | Inventoried assays | length |
| GAPDH | NM_002046.3 | Hs99999905_ml | 122 bp |
| ROCK2 | NM_004850.3 | Hs01071451_ml | 90 bp |

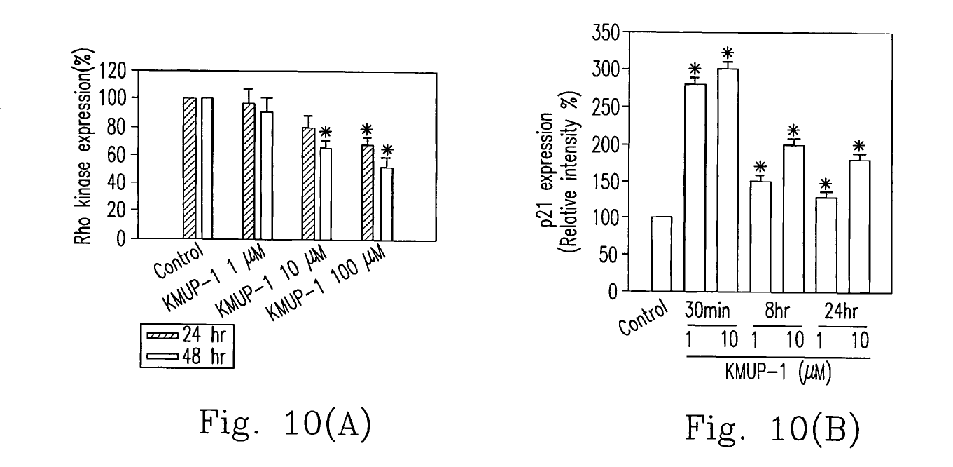

Please refer to FIG. 10(A), which shows the relative gene expression alterations inhibited by KMUP-1 in PZ-HPV-7 cells using GAPDH as internal reference gene. FIG. 10(A) is a quantitative analysis of QRT-PCR, which illustrates the results of Rho kinase mRNA expression in PZ-HPV-7 cells compared with the control after 24 and 48 hrs KMUP-1 treatments. It is found that KMUP-1 (1-100 μM) inhibits Rho kinase gene expression down to 96.00±10.00, 80.00±8.80 and 68.00±5.01% of control after treatment for 24 hours. KMUP-1 (1, 10, 100 μM) inhibits Rho kinase gene expression down to 90.00±10.05, 66.00±5.80 and 52.00±7.01% of control at 48 hours, respectively. The results also show that KMUP-1 inhibits Rho kinase gene expression in a time- and dose-dependent manner in human prostate epithelial cells.

Please refer to FIG. 10(B), which shows the p21 protein expression in PZ-HPV-7 cells analyzed by western blot in the presence of KMUP-1 (1-10 μM) for 30 minutes, 8 hours, and 24 hours respectively. To investigate how KMUP-1 arrests G₀/G₁ cell cycle in human prostate epithelial cells PZ-HPV-7, the western blot is performed to examine the production of CDK inhibitor, p21, in PZ-HPV-7 cells, treated with or without KMUP-1 (1-10 μM) at 30 minutes, 8 hours and 24 hours. The level of p21 protein was compared with levels of β-actin protein. KMUP-1 (1, 10 μM) potently induces the production of p21 protein levels until 24 hours. Based on the above, these results suggest that KMUP-1 arrests cell cycle at G₀/G₁ phase by inducing the expression of p21 protein.

Cyclin/cyclin-dependent kinase (CDK) protein complexes function at different cell cycle stages. p21 protein is a CDK inhibitor known to bind to cyclin-CDK complexes following the anti-mitogenic signals to induce cell cycle arrest at G₀/G₁ phase. p21 protein may be potentially used as a therapeutic target to reduce cell proliferation. The present results suggest the ability of KMUP-1 to inhibit the proliferation of prostate epithelial cells coming through the increase of p21 protein and arrested the cell cycle at G₀/G₁ phase.

Besides, Y-27632, a ROCK2 inhibitor, is able to inhibit the expression of ROCK2, which is usually increased, resulting from various stimulations, including by x-adrenergic agonist phenylephrine. Y-27632 has been described to have anti-proliferation and anti-adrenergic contraction activity in prostatic smooth muscle cells and tissues^{[10]}. In this study, the expression of ROCK2 in rat prostate is certainly induced by phenylephrine. In contrast to Y-27632, KMUP-1 not only inhibits phenylephrine-induced expression of ROCK2, which is a downstream signaling of cGMP/PKG pathway in vascular system^{[25]}, but also inhibits the α_{1A}/α_{1D}-adrenoceptor binding ability in rat prostate smooth muscle. Theoretically, it is reasonable to suggest that KMUP-1 also inhibits cGMP-regulated expression of ROCK2 in human prostatic epithelial cells. Obviously, KMUP-1 can inhibit the growth of human prostate epithelial cells by Rho kinase inhibition, evidenced by expression of p21 protein in epithelial PZ-HPV-7 cells in FIG. 10.

Statistical Analysis

All data are expressed as the mean±SEM. Statistical differences were analyzed by two-tailed unpaired Student's t test respectively. Whenever a control group was compared with more than one treatment group, one-way analysis of variance was used. When the analysis of variance found a statistical difference, results were further analyzed with Dunnett's test. A P value less than 0.05 were considered to significant in all experiments. Analysis of the data and plotting of the figures were performed using statistical software (SigmaPlot version 8.0 and SigmaStat Version 2.03, Chicago, Ill.) run on an IBM-compatible computer.

Clinical use of 5α-reductase inhibitors and α-adrenergic blockers can result in sexual dysfunction or ejaculatory disorders for the treatment of BPH and LUTS. Although PDE5 inhibitors are commonly used to treat erectile dysfunction, PDE inhibitors are also suggested to regulate bladder and prostate smooth muscle tone. Recent researches suggest that the combination of α_{1A} and/or α_{1D}-adrenergic blocker and PDE5 inhibitor may be useful in treating patients having BPH and LUTS-associated erectile dysfunction^{[26, 27]}. In the mentioned studies, it has shown that low concentration of sildenafil (0.1 mM) can sensitize tamsulosin-induced concentration-response relaxation and shift the response curve to the left side. Accordingly, it is strongly suggested that the combination of PDE inhibition with α_{1A} and/or α_{1D}-adrenergic blockade by KMUP-1 might have positive impact in the treatment of patients having BPH/LUTS and associated male erectile dysfunction.

This invention first demonstrates that KMUP-1 has the selective α_{1A}/α_{1D}-adrenergic blocking activity on prostate smooth muscle tone. KMUP-1 multiply possesses PDE inhibition, sGC activation and selective α_{1A}/α_{1D}-adrenergic blocking activity. This is the reason why KMUP-1 can display more extinguish maximal efficacy than tamsulosin in relaxing rat prostate smooth muscle (FIG. 3A).

As mentioned in the above, the present invention indicates that KMUP-1-induced relaxation of the rat prostate is mediated by a mechanism selected from a group consisting of the activation of sGC/cGMP/PKG pathway, a blockage of α_{1A}/α_{1D}-adrenoreceptors, BK_{Ca} and K_{ATP} channels opening, and inhibition of ROCK2 and PDE. Moreover, KMUP-1 not only reduces prostate smooth muscle tone but also inhibits prostatic epithelial cell growth through up-regulating the expression of p21. It is suggested that KMUP-1 can be used by means of altering the prostate contractility, often associated with BPH, LUTS and erectile dysfunction. Accordingly, the present invention firstly shows that a cGMP-enhancer KMUP-1 might be used in the treatment of BPH, and thus it fits the demand of the industry and is industrially valuable.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for treating a patient having a benign prostate hyperplasia, comprising a step of administering to the patient a therapeutically effective amount of one selected from a group consisting of a treating compound of 7-[2-[4-(2-Chlorobenzene)piperazinyl]ethyl]-1,3-dimethyl xanthine, a salt of the treating compound, a solvate of the treating compound and a combination thereof, wherein the compound has an effect on treating the benign prostate hyperplasia via a mechanism selected from a group consisting of an activation of PKG pathway, a blockage of an adrenoceptor, an opening of a potassium channel and an inhibition of PDE activity.

2. A method as claimed in claim 1, wherein the PKG pathway is sGC/cGMP/PKG pathway.

3. A method as claimed in claim 1, wherein the adrenoceptor is an α_{1A}/α_{1D}-adrenergic receptor with a relatively high selectivity.

4. A method as claimed in claim 1, wherein the potassium channel is one selected from a group consisting of a BK_{Ca} channel, a K_{ATP} channel and a combination thereof.

5. A method as claimed in claim 1, wherein the inhibition of PDE is combined with an inhibition of ROCK2.

6. A method for treating a patient having a lower urinary tract symptom, comprising a step of administering to the patient a therapeutically effective amount of one selected from a group consisting of a treating compound of 7-[2-[4-(2-Chlorobenzene)piperazinyl]ethyl]-1,3-dimethyl xanthine, a salt of the treating compound, a solvate of the treating compound and a combination thereof, wherein the compound has an effect on treating the benign prostate hyperplasia via a mechanism selected from a group consisting of an activation of PKG pathway, a blockage of an adrenoceptor, an opening of a potassium channel and an inhibition of PDE activity.

7. A method as claimed in claim 6, wherein the PKG pathway is sGC/cGMP/PKG pathway.

8. A method as claimed in claim 6, wherein the adrenoceptor is an α_{1A}/α_{1D}-adrenergic receptor with a relatively high selectivity.

9. A method as claimed in claim 6, wherein the potassium channel is one selected from a group consisting of a BK_{Ca} channel, a K_{ATP} channel and a combination thereof.

10. A method as claimed in claim 6, wherein the inhibition of PDE is combined with an inhibition of ROCK2.

11. A method for treating a patient having an erectile dysfunction, comprising a step of administering to the patient a therapeutically effective amount of one selected from a group consisting of a treating compound of 7-[2-[4-(2-Chlorobenzene)piperazinyl]ethyl]-1,3-dimethyl xanthine, a salt of the treating compound, a solvate of the treating compound and a combination thereof, wherein the compound has an effect on treating the benign prostate hyperplasia via a mechanism selected from a group consisting of an activation of PKG pathway, a blockage of an adrenoceptor, an opening of a potassium channel and an inhibition of PDE activity.

12. A method as claimed in claim 11, wherein the PKG pathway is sGC/cGMP/PKG pathway.

13. A method as claimed in claim 11, wherein the adrenoceptor is an α_{1A}/α_{1D}-adrenergic receptor with a relatively high selectivity.

14. A method as claimed in claim 11, wherein the potassium channel is one selected from a group consisting of a BK_{Ca} channel, a K_{ATP} channel and a combination thereof.

15. A method as claimed in claim 11, wherein the inhibition of PDE is combined with an inhibition of ROCK2.
